(19) United States
(12) Patent Application Publication
(10) Pub. No.: US 2024/0207040 A1
(43) Pub. Date: Jun. 27, 2024

(54) CONNECTIVE TISSUE AUGMENTATION AND FIXATION DEVICE

(71) Applicant: Biomet Manufacturing, LLC, Warsaw, IN (US)

(72) Inventor: Ryan A. Kaiser, Leesburg, IN (US)

(21) Appl. No.: 18/533,343

(22) Filed: Dec. 8, 2023

Related U.S. Application Data
(60) Provisional application No. 63/434,304, filed on Dec. 21, 2022.

(51) Int. Cl. A61F 2/08

(52) U.S. Cl. CPC A61F 2/0811; A61F 2002/0847; A61F 2002/0876

(57) ABSTRACT

System and techniques for connective tissue augmentation and fixation device are described herein. The connective tissue augmentation and fixation device can include a central body, a first side tail, and a second side tail. The central body can be configured to at least partially surround a graft. The central body can extend longitudinally between a first end and a second end. The first side tail can extend from the first end of the central body. The first side tail can be configured to attach to a first bone of a patient. The second side tail can extend from the second end of the central body. The second side tail can be configured to attach to a second bone of the patient.

### CLAIM OF PRIORITY

This application claims the benefit of U.S. Provisional Patent Application Ser. No. 63/434,304, filed on Dec. 21, 2022, the benefit of priority of which is claimed hereby, and which is incorporated by reference herein in its entirety.

### TECHNICAL FIELD

Embodiments described herein generally relate to connective tissue repairs and, more specifically, a connective tissue augmentation and fixation device.

### BACKGROUND

Ligaments and tendons are crucial components of the musculoskeletal system. The main ligaments are the cranial cruciate ligament (CCL), posterior cruciate ligament (PCL), lateral collateral ligament (LCL), and medial collateral ligament (MCL). These ligaments and tendons keep joints stable and are often strained or injured due to a fall, a car accident, a sports injury, or trauma to the knee or ankle. Damage to a joint caused by such injuries is often difficult to repair and is often a source of significant discomfort and inconvenience to the patient. Tendons connect muscles to bones. A tear or injury to a tendon usually involves separating the tendon from the bone. It is often difficult to repair these tears or injuries.

To treat an injury to a tendon or ligament, it is often necessary to fuse the torn or damaged tendon with other healthy portions of the tendon to stabilize the injury and prevent further damage to the tendon or ligament. This can be done surgically, but can also be performed using biologic tissue grafts or biologically compatible implants to provide stability to the tendon or ligament and promote healing of the injured tendon or ligament.

### BRIEF DESCRIPTION OF THE DRAWINGS

In the drawings, which are not necessarily drawn to scale, like numerals may describe similar components in different views. Like numerals having different letter suffixes may represent different instances of similar components. The drawings illustrate generally, by way of example, but not by way of limitation, various embodiments discussed in the present document.

FIG. 1 illustrates an example of a connective tissue augmentation and fixation device installed within a patient, according to an embodiment.

FIG. 2 illustrates a schematic of an example of a connective tissue augmentation and fixation device, according to an embodiment.

FIG. 3 illustrates a schematic of an example of a connective tissue augmentation and fixation device, according to an embodiment.

FIG. 4A illustrates a schematic of an example of a connective tissue augmentation and fixation device, according to an embodiment.

FIG. 4B illustrates an enlarged view of portion 4B-4B of the connective tissue augmentation and fixation device from FIG. 4A, according to an embodiment.

FIG. 5 illustrates a schematic of an example of a connective tissue augmentation and fixation device, according to an embodiment.

FIG. 6 illustrates a schematic of an example of a connective tissue augmentation and fixation device, according to an embodiment.

FIG. 7 illustrates a schematic of an example of a connective tissue augmentation and fixation device, according to an embodiment.

FIG. 8 illustrates a flow diagram of an example of a method for connective tissue augmentation and fixation device, according to an embodiment.

### DETAILED DESCRIPTION

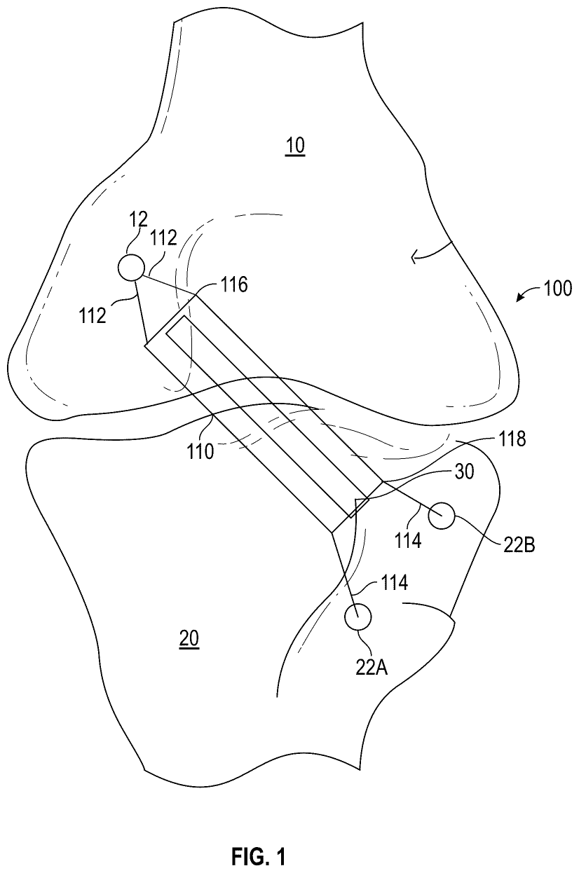

The inventor of the present disclosure has identified at least the following problems with ligament reconstructive surgeries: 1) poor donor graft; 2) poor patient bone quality; 3) the graft being damaged from the fixation device; 4) fixation methods inhibit graft regrowth; and 5) poor device tendon interface. The present disclosure discusses a connective tissue augmentation and fixation device designed to protect and support a graft, ligament, tendon, muscle, or the like, during implantation by incorporating fixation capabilities into the device. Fixation of tendon and ligament repairs and reconstruction with devices that enhance the initial healing response can allow for superior graft integration and improve patient outcomes. The connective tissue augmentation and fixation device will be discussed below with reference to FIGS. 1-8.

FIG. 1 illustrates an example of a connective tissue augmentation and fixation device (device 100) being installed within a patient. The device 100 can be configured to aid in the augmentation and fixation of a muscle, ligament, tendon, graft, or the like (graft 30) within the patient. In examples, the device 100 can be used to repair a partial tear of a tendon or ligament. In another example, the device 100 can be used to repair a complete tear of a tendon or a ligament. The device 100 can be made from a bioabsorbable material such that the device 100 provides support after the device 100 is installed within the patient, but the device 100 dissolves over time to permit muscle and tendon growth to strengthen the joint using the anatomy of the patient. In another example, the device 100 can be made from polyethylene. In yet another example, the device 100 can be made from polyethylene, any biocompatible material, any bioabsorbable material, any combination thereof, or the like. The device 100 can include a central body 110, a first side tail 112, and a second side tail 114. The central body 110 can include a first end 116 and a second end 118.

The central body 110 can at least partially surround the graft 30. In examples, the central body 110 can extend longitudinally between the first end 116 and the second end 118. In examples, the central body 110 can be generally cylindrically shaped such that the central body 110 can slide around the graft 30 to surround the graft 30. In another example, the central body 110 can be flat such that the central body 110 can be wrapped around the graft 30 to surround at least a portion of the graft 30.

The first side tail 112 can extend from the first end 116 of the central body 110. The first side tail 112 can be configured to attach to a first bone of a patient (first bone 10). For example, the first side tail 112 can fit within a channel 12 formed within the first bone 10. The channel 12 can be formed by a surgeon during the operation before the insertion of the device 100. In other examples, the first side tail 112 can secure the device 100 to an anchor, attachment device, or the like, installed within the channel 12 of the first bone 10. The first side tail 112 can be designed such that tension can be transferred from the first side tail 112 to the central body 110 to support the device 100 after implantation into the patient.

The second side tail 114 can extend from the second end 118 of the central body 110. The second side tail 114 can be configured to attach to a second bone of the patient (second bone 20). For example, the second side tail 114 can fit within a channel 22 formed within the second bone 20. The channel 22 can be formed by a surgeon during the operation and before the insertion of the device 100. In other examples, the second side tail 114 can secure the device 100 to an anchor, attachment device, or the like, installed within the channel 22 of the second bone 20. The second side tail 114 can be designed such that tension can be transferred from the first side tail 112 to the central body 110 to support the device 100 after implantation into the patient.

As shown in FIG. 1, the device 100 can be configured such as to permit two first side tails 112 to route through a single channel 12. The device 100 can also be configured such as to permit each of the second side tails 114 to a separate channel. For example, a first second side tail 114 can be routed to channel 22A and a second second side tail 114 can be routed to channel 22B. In another example, any number of tails (112 or 114) can be configured to be routed through any number of channels (12 or 22) to secure the device 100 to the first bone 10 and the second bone 20, respectively.

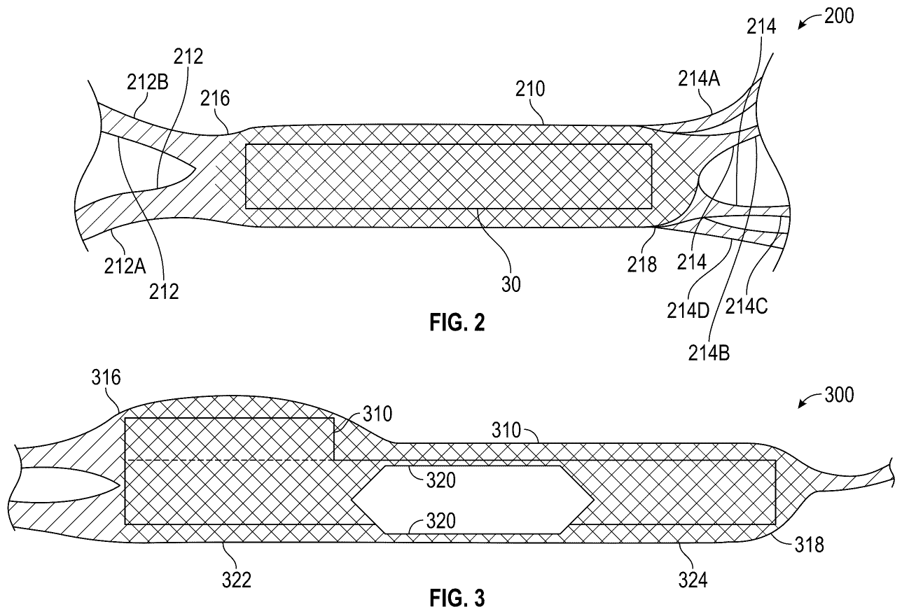

FIG. 2 illustrates a schematic of an example of a connective tissue augmentation and fixation device (device 200), according to an embodiment. The device 200 can surround the graft 30 and be implanted into a patient to aid in the augmentation and fixation of the graft 30. In an example, the device 200 can be used to repair a complete tear of a tendon or a ligament. The device 200 can be made from a bioabsorbable material such that the device 200 provides support after the device 200 is installed within the patient, and the device 200 dissolves over time to permit muscle and tendon growth to strengthen the joint using the anatomy of the patient. In another example, the device 200 can be made from polyethylene. In yet another example, the device 200 can be made from polyethylene, any biocompatible material, any bioabsorbable material, any combination thereof, or the like. The device 200 can include a central body 210, a first side tail 212, and a second side tail 214. The central body 210 can include a first end 216 and a second end 218.

As shown in FIG. 2, the central body 210 can completely surround the graft 30. The central body 210 can generally be a hollow cylinder that permits the device 200 to slide around the graft 30. The central body 210 can extend between the first end 216 and the second end 218.

As shown in FIG. 2, the first side tail 212 can extend from the first end 216 of the central body 210. As shown in FIG. 3, a single side tail can extend from a side of the central body. In yet another example, any number of side tails can extend from the central body.

In an example, a first side tail 212A and a first side tail 212B can extend from the first end 216 of the central body 210. The first side tail 212A can extend from the central body 210 to a first channel (e.g., the channel 12A in FIG. 1) to attach within the channel 12A or to an anchor, attachment mechanism, or the like, installed within the channel 12A. The first side tail 212B can extend from the central body 210 to a first channel (e.g., the channel 12B in FIG. 1) to attach within the channel 12B or to an anchor, attachment mechanism, or the like, installed within the channel 12B. Thus, the channels 12A and 12B can be installed within the bone 10 such that the first side tail 212A and the first side tail 212B can optimize the support for the device 200. In another example, any number of the first side tail 212 can be matched with any number of the channels to provide support necessary to secure the device 200 to the first bone. For example, the first side tail 212A and the first side tail 212B can both route to the channel 12A to attach to the bone of the patient.

As shown in FIG. 2, the second side tail 214 can extend from the second end 218 of the central body 210. A second side tail 214A, a second side tail 214B, a second side tail 214C, and a second side tail 214D extend from the first end 216 of the central body 210. In examples, the second side tail 214A can extend from the central body 210 to a second channel (e.g., the channel 22A in FIG. 1) to attach within the channel 22A or to an anchor, attachment mechanism, or the like, installed within the channel 22A. The second side tail 214B can extend from the central body 210 to a first channel (e.g., the channel 22B in FIG. 1) to attach within the channel 22B or to an anchor, attachment mechanism, or the like, installed within the channel 22B. Moreover, the second side tail 214C can extend from the central body 210 to a third channel to attach within the channel or to an anchor, attachment mechanism, or the like, installed within the channel. The second side tail 214D can extend from the central body 210 to a third channel to attach within the channel or to an anchor, attachment mechanism, or the like, installed within the channel. Thus, the channels can be installed within the bone such that the second side tail 214A, second side tail 214B, second side tail 214C, and second side tail 214D can optimize support for the device 200. In another example, any number of the second side tail 214 can be matched with any number of the channels 12 to provide support necessary to secure the device 200 to the second bone.

FIG. 3 illustrates a schematic of an example of a connective tissue augmentation and fixation device (device 300). The device 300 can surround the graft 30 and be implanted into a patient to aid in the augmentation and fixation of the graft 30. In an example, the device 300 can be used to repair a complete tear of a tendon or a ligament. The device 300 can be made from a bioabsorbable material such that the device 300 provides support after the device 300 is installed within the patient, and the device 300 dissolves over time to permit muscle and tendon growth to strengthen the joint using the anatomy of the patient. In another example, the device 300 can be made from polyethylene. In yet another example, the device 300 can be made from polyethylene, any biocompatible material, any bioabsorbable material, any combination thereof, or the like.

As shown in FIG. 3, the device 300 can include a central body 310 that can include a bifurcation 320. The bifurcation 320 can be located in the central body 310 between a first end 316 and a second end 318. In some examples, the central body 310 can include a first section 322 and a second section 324. The first section 322 and the second section 324 can come together at the bifurcation 320.

The bifurcation 320 can permit access to the central body 310 such that a graft, muscle, tendon, ligament, or the like, can more easily be adjusted within the central body 310 after the device 300 is placed around the graft, muscle, tendon, ligament, or the like. For example, the first section 322 can be placed around a muscle, tendon, ligament, or the like, that is still at least partially attached to a bone of the patient, and the second section 324 can be placed around a torn muscle, tendon, ligament, or the like, that is detached from the bone of the patient. In another example, the second section 324 can be placed around a graft that can be used to heal the muscle, tendon, ligament, or the like within the first section 322. The bifurcation 320 also decrease an amount of material to make the device 300. The decreased material of the device 300 can reduce manufacturing costs and improve healing of the joint after implantation.

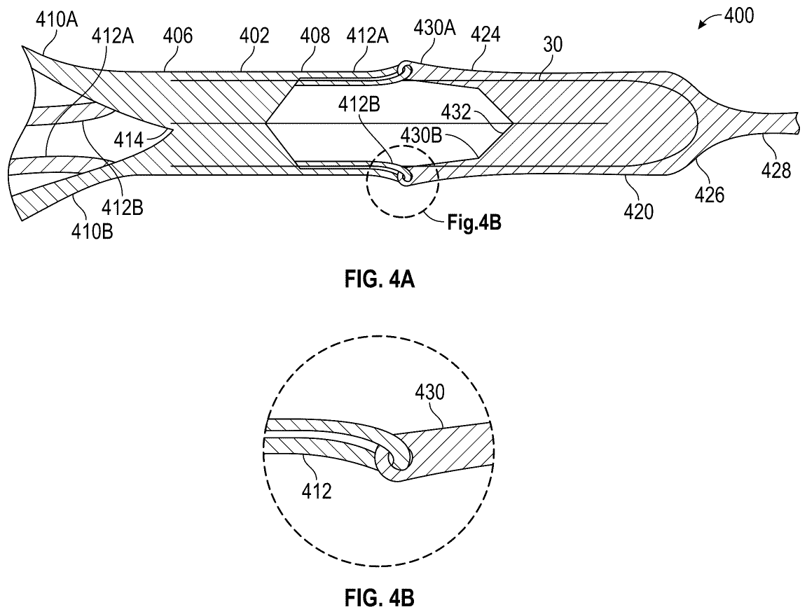

FIGS. 4A and 4B will be discussed together below. FIG. 4A illustrates a schematic of an example of a connective tissue augmentation and fixation device (device 400). FIG. 4B illustrates an enlarged view of portion 4B-4B of the device 400 from FIG. 4A.

The device 400 can surround the graft 30 and be implanted into a patient to aid in the augmentation and fixation of the graft 30. In an example, the device 400 can be used to repair a complete or partial tear of a tendon or a ligament. The device 400 can be made from a bioabsorbable material such that the device 400 provides support after the device 400 is installed within the patient, and the device 400 dissolves over time to permit muscle and tendon growth to strengthen the joint using the anatomy of the patient. In another example, the device 400 can be made from polyethylene. In yet another example, the device 400 can be made from polyethylene, any biocompatible material, any bioabsorbable material, any combination thereof, or the like.

The device 400 can include a first portion 402 extending longitudinally between a first end 406 and a second end 408. The first portion 402 can include a first tail 410 and a second tail 412.

The first tail 410 can secure the first portion 402 of the device 400 to a first bone of the patient. The first tail 410 can include a hook, clamp, attachment mechanism, or the like to attach the first portion 402 to the first bone of the patient. In an example, the first tail 410 can be securable to a post, attachment mechanism, or the like that is secured to the first bone to secure the first portion 402 to the first bone. The first tail 410 can extend from the first end 406 of the first portion 402. In examples, a first tail 410A and a first tail 410B can extend from the first end 406 of the device 400. As shown in FIG. 4A, the first tail 410A and the first tail 410B can both have a bifurcation. In an example, either the first tail 410A or the first tail 410B can have one or more bifurcations. The first portion 402 can include any number of the first tail 410 to secure the first portion 402 to the first bone of a patient.

The second tail 412 can secure the first portion 402 of the device 400 to a second portion 420 of the device 400. The second tail 412 can extend from the second end 408 of the first portion 402. In examples, the first portion 402 can include a second tail 412A and a second tail 412B. In another example, the first portion 402 can include any number of the second tail 412 to secure the first portion 402 of the device 400 to the second portion 420 of the device 400.

The first portion 402 can define a first cavity 414. The first cavity 414 can extend from the first end 406 and the second end 408 of the first portion 402. The first cavity 414 can receive a graft, muscle, tendon, ligament, or the like. In examples, the graft, muscle, tendon, ligament, or the like can be secured within the first cavity 414 with a stitching or any other form of fastening. In another example, the graft, muscle, tendon, ligament, or the like can be secured within the first cavity 414 by the friction applied to the first tail 410 and the second tail 412.

The device 400 can also include the second portion 420. The second portion 420 can extend longitudinally between a first end 424 and a second end 426. The second portion can include a third tail 428 and a hooks 430.

The third tail 428 can secure the device 400 to a second bone of a patient. The third tail 428 can include a hook, clamp, attachment mechanism, or the like to attach the second portion 420 to the second bone. In another example, the third tail 428 can be securable to a post, attachment mechanism, or the like, attached to the second bone to secure the second portion 420 to the second bone. The third tail 428 can extend from the second end 426 of the second portion 420. In examples, the third tail 428 can include bifurcations. The second portion 420 can include any number of the third tail 428 to secure the second portion 420 to the second bone.

The second portion 420 can also include hooks 430. The hooks 430 can extend from the first end 424 of the second portion 420. The hooks 430 can be configured to receive the second tail 412 of the first portion 402 to couple the first portion 402 and the second portion 420. In examples, the second portion 420 can include a hook 430A and a hook 430B. The hooks 430A and 430B can extend from the first end 424 of the second portion 420 to receive a second tail 412A and 412B, respectively.

In an example, the second tail 412 of the first portion 402 can extend through the hooks 430 of the second portion 420 and back through the first cavity 414 towards the first end 406 of the first portion 402. Here, the second tail 412 can be secured to the first tail 410. In another example, the second tail 412 can include a hook, clamp, or attachment mechanism to attach to the first bone. In yet another example, the second tail 412 can be securable to a post, attachment mechanism, or the like, that is attached to the first bone to secure the first portion 402 to the first bone of the patient. Moreover, tension can be applied to the second tail 412 to tighten the engagement between the first portion 402 and the second portion 420 of the device 400. Such tightening can apply tension through the first cavity 414 and a second cavity 432 to secure the graft, muscle, tendon, ligament, or the like within the first cavity 414 and the second cavity 432.

In an example, the second portion 420 can define the second cavity 432. The second cavity 432 can extend between the first end 424 and the second end 426 of the second portion 420. The second portion 420 can receive a graft, muscle, tendon, ligament, or the like. In examples, the graft, muscle, tendon, ligament, or the like can be secured within the second portion 420 with stitching or any other form of fastening. In another example, the graft, muscle, tendon, ligament, or the like can be secured within the second portion 420 by the friction applied to the third tail 428 and the hooks 430.

In an operative example of the device 400, a first graft, muscle, tendon, ligament, or the like can be placed within the first cavity 414 and a second graft, muscle, tendon, ligament, or the like can be place within the second portion 420. In examples, the grats, muscles, tendons, ligaments, or the like, can be secured within the first cavity 414 and the second portion 420 with a stitching or any other attachment. As tension is applied to the first tail 410 to attach the first tail 410 to the first bone, the graft, muscle, tendon, ligament, or the like within the first cavity 414 and the muscle, tendon, ligament, or the like within the second portion 420 can move toward each other. The tension applied to the first tail first tail 410 can help hold the graft, ligament, muscle, tendon, or the like within the first cavity 414. The tension applied to the first tail 410 can also be transferred to the second portion 420 via the hooks 430. Thus, the tension applied to the first tail 410 can also help hold the graft, ligament, tendon, muscle, or the like within the second cavity 432.

In the example of the device 400 shown in FIG. 4A, the device 400 is asymmetrical such that a different number of tails extend from each portion of the device 400. In another example, the device 400 can be symmetrical. For example, the tails that attach to the second end 426 of the second portion 420 can match the tails that attach to the first end 406 of the first portion 402.

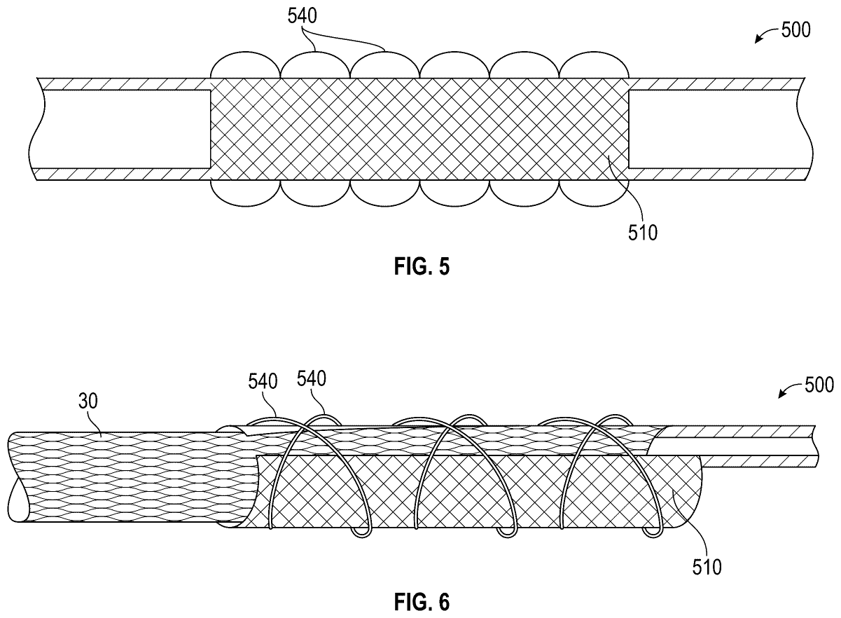

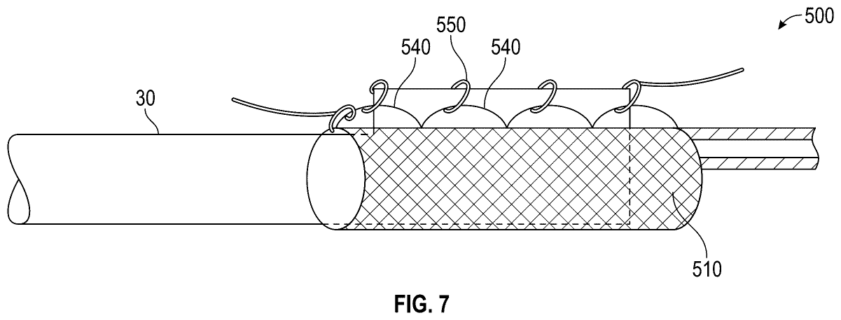

FIGS. 5-7 will be discussed together below. FIG. 5 illustrates a schematic of an example of a connective tissue augmentation and fixation device (device 500). FIG. 6 illustrates a schematic of another example of the device 500. FIG. 7 illustrates a schematic of yet another example of the device 500.

The device 500 can surround the graft 30 and be implanted into a patient to aid in the augmentation and fixation of the graft 30. In an example, the device 500 can be used to repair a complete or partial tear of a tendon or a ligament. The device 500 can be made from a bioabsorbable material such that the device 500 provides support after the device 500 is installed within the patient, and the device 500 dissolves over time to permit muscle and tendon growth to strengthen the joint using the anatomy of the patient. In another example, the device 500 can be made from polyethylene. In yet another example, the device 500 can be made from polyethylene, any biocompatible material, any bioabsorbable material, any combination thereof, or the like.

As shown in FIG. 5, the central body 510 can be generally rectangular such that the device 500 can lie flat and wrap around a graft, ligament, tendon, muscle or the like. As the central body 510 is wrapped around the graft, ligament, tendon, muscle, or the like, the central body 510 can be stitched or attached to the graft, ligament, tendon, muscle, or the like to secure the central body 510.

In some examples, a central body 510 of the device 500 can include two or more hooks (hooks 540) around a periphery of the central body 510. The central body 510 can include the hooks 540 around an entire periphery of the central body 510. The hooks 540 can come together, or move towards each other, as the central body 510 is wrapped around a graft, ligament, muscle, tendon, or the like. The hooks 540 can then be stitched to the graft, ligament, muscle, tendon, or the like, to secure the device 500 to the graft, ligament, muscle, tendon, or the like. In another example, the hooks 540 can be attached to each other as the central body 510 surrounds the graft, ligament, muscle, tendon, or the like.

In some examples, the device 500 can include a tensioning line 550 configured to engage with the hooks 540 as the central body 510 at least partially surrounds the graft, ligament, muscle, tendon, or the like. The tensioning line 550 can be tightened to secure the device 500 on the graft, ligament, tendon, muscle, or the like.

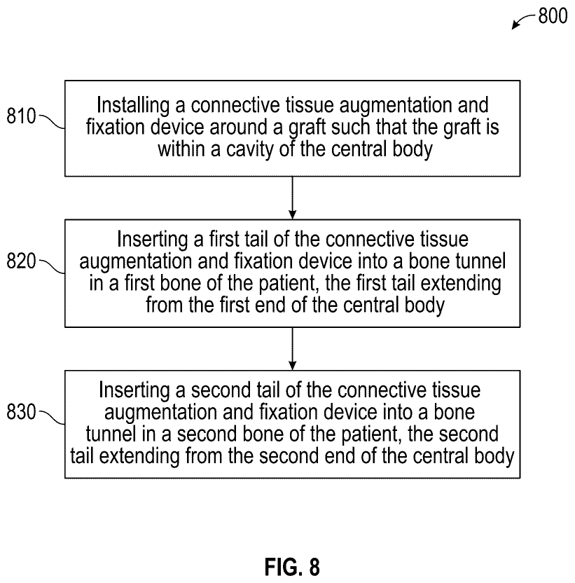

FIG. 8 illustrates a schematic view of the method 800, in accordance with at least one example of this disclosure. The method 800 can be a method of connective tissue augmentation and fixation device. More specific examples of the method 800 are discussed below. The steps or operations of the method 800 are illustrated in a particular order for convenience and clarity; many of the discussed operations can be performed in a different sequence or in parallel without materially impacting other operations. The method 800 as discussed can include operations performed by multiple different actors, devices, or systems. It is understood that subsets of the operations discussed in the method 800 can be attributable to a single actor, device, or system could be considered a separate standalone process or method.

At procedure 810, the method 800 can include installing the connective tissue augmentation and fixation device (e.g., the device 100, the device 200, the device 300, the device 400, or the device 500) around a graft such that the graft can be within a cavity of a central body. In examples, the device can also be used to repair at least partially torn muscle, tendon, ligament, or the like. The graft can be secured to the device via stitching, a clip, a combination of temporary attachments, or the like. The graft and the device can be attached to one or more bones of a patient.

At procedure 820, the method 800 can include inserting a first tail of the connective tissue augmentation and fixation device (e.g., the device 100, the device 200, the device 300, the device 400, or the device 500) into a bone tunnel in a first bone of the patient, the first tail extending from the first end of the central body.

At procedure 830, the method 800 can include inserting a second tail of the connective tissue augmentation and fixation device (e.g., the device 100, the device 200, the device 300, the device 400, or the device 500), into a bone tunnel in a second bone of the patient. The second tail can extend from the second end of the central body.

### Additional Notes & Examples

Example 1 is a connective tissue augmentation and fixation device comprising: a central body configured to at least partially surround a graft, the central body extending longitudinally between a first end and a second end; a first side tail extending from the first end of the central body and configured to attach to a first bone of a patient; and a second side tail extending from the second end of the central body and configured to attach to a second bone of the patient.

In Example 2, the subject matter of Example 1 can include, wherein the central body comprises: a bifurcation that permits access to the graft within the connective tissue augmentation and fixation device, the bifurcation located between the first end and the second end of the central body.

In Example 3, the subject matter of Example 2 can include, wherein the central body can include a first section and a second section, the first section and the second section are joined at the bifurcation.

In Example 4, the subject matter of Example 3 can include, wherein the first section and the second section are removably attached at the bifurcation.

In Example 5, the subject matter of Examples 1-4 can include, wherein the central body can include two or more hooks around a periphery of the central body.

In Example 6, the subject matter of Example 5 can include, wherein the two or more hooks move towards each other as the central body surrounds the graft to secure to one another and hold the connective tissue augmentation and fixation device on the graft.

In Example 7, the subject matter of Examples 5-6 can include, a tensioning line configured to engage with the two or more hooks as the central body partially surrounds the graft, the tensioning line can be tightened to secure the connective tissue augmentation and fixation device on the graft.

In Example 8, the subject matter of Examples 1-7 can include, wherein at least a portion of the connective tissue augmentation and fixation device is made of a bioabsorbable material.

In Example 9, the subject matter of Examples 1-8 can include, wherein a first portion of the connective tissue augmentation and fixation device is polyethylene, and a second portion is a bioabsorbable material.

Example 10 is a connective tissue augmentation and fixation device comprising: a first portion extending longitudinally between a first end and a second end, the first portion including: a first tail extending from the first end of the first portion; and a second tail extending from the second end of the first portion; and a second portion extending longitudinally between a first end and a second end, the second portion including: a third tail extending from the second end of the second portion; and a hook extending from the first end of the second portion, the hook configured to receive the second tail of the first portion to couple the first portion and the second portion.

In Example 11, the subject matter of Example 10 can include, wherein the first portion defines a first cavity extending from the first end to the second end of the first portion.

In Example 12, the subject matter of Example 11 can include, wherein the second tail of the first portion extends through the hook of the second portion and back through the first cavity towards the first end of the first portion.

In Example 13, the subject matter of Example 12 can include, wherein the second portion defines a second cavity extending between the first end and the second end of the second portion, wherein the first cavity and the second cavity are each configured to receive a graft.

In Example 14, the subject matter of Example 13 can include, wherein the first tail and the second tail of the first portion and the third tail of the second portion are each configured to be attached to a bone of a patient.

In Example 15, the subject matter of Example 14 can include, wherein the first tail and the second tail of the first portion are configured to be attached to a first bone of the patient, and wherein the third tail of the second portion is configured to be attached to a second bone of a patient.

Example 16 is a method of implanting a connective tissue augmentation and fixation device into a patient, the connective tissue augmentation and fixation device can include, a central body extending between a first end and a second end and defining a cavity extending between the first end and the second end, the method comprising: installing the connective tissue augmentation and fixation device around a graft such that the graft is within the cavity of the central body; inserting a first tail of the connective tissue augmentation and fixation device into a bone tunnel in a first bone of the patient, the first tail extending from the first end of the central body; and inserting a second tail of the connective tissue augmentation and fixation device into a bone tunnel in a second bone of the patient, the second tail extending from the second end of the central body.

In Example 17, the subject matter of Example 16 can include, wherein installing the connective tissue augmentation and fixation device around the graft further comprises: stitching one or more connective stitches between the connective tissue augmentation and fixation device and the graft to hold the graft within the cavity of the central body.

In Example 18, the subject matter of Examples 16-17 can include, wherein installing the connective tissue augmentation and fixation device around the graft further comprises: inserting a first portion of a torn connective tissue of the patient within a first section of the cavity, the first section of the cavity defined by a first segment of the connective tissue augmentation and fixation device; securing the first portion of the torn connective tissue to the connective tissue augmentation and fixation device to hold the first portion of the torn connective tissue within the first section of the cavity; inserting a second portion of the torn connective tissue of the patient within a second section of the cavity, the second section of the cavity defined by a second segment of the connective tissue augmentation and fixation device; and securing the second portion of the torn connective tissue to the connective tissue augmentation and fixation device to hold the second portion of the torn connective tissue within the second section of the cavity.

In Example 19, the subject matter of Example 18 can include, wherein the first portion of the connective tissue augmentation and fixation device can include a first tail extending from a first end of the first portion of the connective tissue augmentation and fixation device and a second tail extending from a second end of the first portion of the connective tissue augmentation and fixation device, and wherein the second portion of the connective tissue augmentation and fixation device can include a hook extending from a first end of the second portion of the connective tissue augmentation and fixation device and a third tail extending from a second end of the second portion of the connective tissue augmentation and fixation device.

In Example 20, the subject matter of Example 19 can include, securing the first portion of the connective tissue augmentation and fixation device to the second portion of the connective tissue augmentation and fixation device by threading the second tail through the hook; pulling the second tail through the hook; and anchoring the second tail into a bone channel of the first bone of the patient.

Example 21 is an apparatus comprising means to implement of any of Examples 1-20.

Example 22 is a system to implement of any of Examples 1-20.

Example 23 is a method to implement of any of Examples 1-20.

The above-detailed description can include references to the accompanying drawings, which form a part of the detailed description. The drawings show, by way of illustration, specific embodiments that may be practiced. These embodiments are also referred to herein as “examples.” Such examples may include elements in addition to those shown or described. However, the present inventors also contemplate examples in which only those elements shown or described are provided. Moreover, the present inventors also contemplate examples using any combination or permutation of those elements shown or described (or one or more aspects thereof), either with respect to a particular example (or one or more aspects thereof), or with respect to other examples (or one or more aspects thereof) shown or described herein.

All publications, patents, and patent documents referred to in this document are incorporated by reference herein in their entirety, as though individually incorporated by reference. In the event of inconsistent usages between this document and those documents so incorporated by reference, the usage in the incorporated reference(s) should be considered supplementary to that of this document; for irreconcilable inconsistencies, the usage in this document controls.

In this document, the terms “a” or “an” are used, as is common in patent documents, to include one or more than one, independent of any other instances or usages of “at least one” or “one or more.” In this document, the term “or” is used to refer to a nonexclusive or, such that “A or B” includes “A but not B,” “B but not A,” and “A and B,” unless otherwise indicated. In the appended claims, the terms “including” and “in which” are used as the plain-English equivalents of the respective terms “comprising” and “wherein.” Also, in the following claims, the terms “including” and “comprising” are open-ended, that is, a system, device, article, or process that includes elements in addition to those listed after such a term in a claim are still deemed to fall within the scope of that claim. Moreover, in the following claims, the terms “first,” “second,” and “third,” etc. are used merely as labels, and are not intended to impose numerical requirements on their objects.

The above description is intended to be illustrative, and not restrictive. For example, the above-described examples (or one or more aspects thereof) may be used in combination with each other. Other embodiments may be used, such as by one of ordinary skill in the art upon reviewing the above description. The Abstract is to allow the reader to quickly ascertain the nature of the technical disclosure and is submitted with the understanding that it will not be used to interpret or limit the scope or meaning of the claims. Also, in the above Detailed Description, various features may be grouped together to streamline the disclosure. This should not be interpreted as intending that an unclaimed disclosed feature is essential to any claim. Rather, inventive subject matter may lie in less than all features of a particular disclosed embodiment. Thus, the following claims are hereby incorporated into the Detailed Description, with each claim standing on its own as a separate embodiment. The scope of the embodiments should be determined with reference to the appended claims, along with the full scope of equivalents to which such claims are entitled.

1. A connective tissue augmentation and fixation device comprising:
a central body configured to at least partially surround a graft, the central body extending longitudinally between a first end and a second end;
a first side tail extending from the first end of the central body and configured to attach to a first bone of a patient; and
a second side tail extending from the second end of the central body and configured to attach to a second bone of the patient.

2. The connective tissue augmentation and fixation device of claim 1, wherein the central body comprises:
a bifurcation that permits access to the graft within the connective tissue augmentation and fixation device, the bifurcation located between the first end and the second end of the central body.

3. The connective tissue augmentation and fixation device of claim 2, wherein the central body includes a first section and a second section, the first section and the second section are joined at the bifurcation.

4. The connective tissue augmentation and fixation device of claim 3, wherein the first section and the second section are removably attached at the bifurcation.

5. The connective tissue augmentation and fixation device of claim 1, wherein the central body includes two or more hooks around a periphery of the central body.

6. The connective tissue augmentation and fixation device of claim 5, wherein the two or more hooks move towards each other as the central body surrounds the graft to secure to one another and hold the connective tissue augmentation and fixation device on the graft.

7. The connective tissue augmentation and fixation device of claim 5, further comprising:
a tensioning line configured to engage with the two or more hooks as the central body partially surrounds the graft, the tensioning line can be tightened to secure the connective tissue augmentation and fixation device on the graft.

8. The connective tissue augmentation and fixation device of claim 1, wherein at least a portion of the connective tissue augmentation and fixation device is made of a bioabsorbable material.

9. The connective tissue augmentation and fixation device of claim 1, wherein a first portion of the connective tissue augmentation and fixation device is polyethylene, and a second portion is a bioabsorbable material.

10. A connective tissue augmentation and fixation device comprising:
a first portion extending longitudinally between a first end and a second end, the first portion including:
a first tail extending from the first end of the first portion; and
a second tail extending from the second end of the first portion; and
a second portion extending longitudinally between a first end and a second end, the second portion including:
a third tail extending from the second end of the second portion; and
a hook extending from the first end of the second portion, the hook configured to receive the second tail of the first portion to couple the first portion and the second portion.

11. The connective tissue augmentation and fixation device of claim 10, wherein the first portion defines a first cavity extending from the first end to the second end of the first portion.

12. The connective tissue augmentation and fixation device of claim 11, wherein the second tail of the first portion extends through the hook of the second portion and back through the first cavity towards the first end of the first portion.

13. The connective tissue augmentation and fixation device of claim 12, wherein the second portion defines a second cavity extending between the first end and the second end of the second portion, wherein the first cavity and the second cavity are each configured to receive a graft.

14. The connective tissue augmentation and fixation device of claim 13, wherein the first tail and the second tail of the first portion and the third tail of the second portion are each configured to be attached to a bone of a patient.

15. The connective tissue augmentation and fixation device of claim 14, wherein the first tail and the second tail of the first portion are configured to be attached to a first bone of the patient, and wherein the third tail of the second portion is configured to be attached to a second bone of a patient.

16. A method of implanting a connective tissue augmentation and fixation device into a patient, the connective tissue augmentation and fixation device includes a central body extending between a first end and a second end and defining a cavity extending between the first end and the second end, the method comprising:
installing the connective tissue augmentation and fixation device around a graft such that the graft is within the cavity of the central body;
inserting a first tail of the connective tissue augmentation and fixation device into a bone tunnel in a first bone of the patient, the first tail extending from the first end of the central body; and
inserting a second tail of the connective tissue augmentation and fixation device into a bone tunnel in a second bone of the patient, the second tail extending from the second end of the central body.

17. The method of claim 16, wherein installing the connective tissue augmentation and fixation device around the graft further comprises:
stitching one or more connective stitches between the connective tissue augmentation and fixation device and the graft to hold the graft within the cavity of the central body.

18. The method of claim 16, wherein installing the connective tissue augmentation and fixation device around the graft further comprises:
inserting a first portion of a torn connective tissue of the patient within a first section of the cavity, the first section of the cavity defined by a first segment of the connective tissue augmentation and fixation device;
securing the first portion of the torn connective tissue to the connective tissue augmentation and fixation device to hold the first portion of the torn connective tissue within the first section of the cavity;
inserting a second portion of the torn connective tissue of the patient within a second section of the cavity, the second section of the cavity defined by a second segment of the connective tissue augmentation and fixation device; and
securing the second portion of the torn connective tissue to the connective tissue augmentation and fixation device to hold the second portion of the torn connective tissue within the second section of the cavity.

19. The method of claim 18, wherein the first portion of the connective tissue augmentation and fixation device includes a first tail extending from a first end of the first portion of the connective tissue augmentation and fixation device and a second tail extending from a second end of the first portion of the connective tissue augmentation and fixation device, and wherein the second portion of the connective tissue augmentation and fixation device includes a hook extending from a first end of the second portion of the connective tissue augmentation and fixation device and a third tail extending from a second end of the second portion of the connective tissue augmentation and fixation device.

20. The method of claim 19, further comprising:
securing the first portion of the connective tissue augmentation and fixation device to the second portion of the connective tissue augmentation and fixation device by threading the second tail through the hook;
pulling the second tail through the hook; and
anchoring the second tail into a bone channel of the first bone of the patient.
